(19) United States
(12) Patent Application Publication
(10) Pub. No.: US 2011/0091531 A1
(43) Pub. Date: Apr. 21, 2011

(54) Cooling Composition

(75) Inventors: Stefan Michael Furrer, Cincinnati, OH (US); Karen Ann Bell, Loveland, OH (US); Fabio Campanile, Zollikerberg (CH); Aloysius Lambertus Doorn, Zeewolde (NL); Joshua Andrew Hagen, Cincinnati, OH (US)

(73) Assignee: GIV AUDAN SA, Vernier (CH)

(21) Appl. No.: 12/992,375

(22) PCT Filed: May 19, 2009
(86) PCT No.: PCT/CH2009/000161

§ 371 Date: Dec. 21, 2010

Related U.S. Application Data
(60) Provisional application No. 61/055,363, filed on May 22, 2008.

(51) Int. Cl. A61K 31/277; A61K 31/16; A61K 31/215; A61K 31/44; A61K 9/127; A61K 9/14; A61K 8/42; A61K 8/37; A61K 8/49; A61K 8/40; A61P 17/02; A61P 11/00; A61Q 11/00; A61Q 19/00; A23L 1/22

(52) U.S. Cl. 424/450; 514/521; 514/629; 514/546; 514/357; 424/484; 424/49; 426/650

(57) ABSTRACT

A liquid cooling composition, which is a mixture of at least one primary cooling compound, at least one different secondary cooling compound and at least one ingestible non-polar solvent for the primary cooling compound, the weight ratios of primary cooling compound:secondary cooling compound:solvent being 1:1.5-2.25:2-4.4.

This disclosure relates to mixtures of cooling compounds for use in practical formulations.

Cooling compounds, that is, chemical compounds that impart a cooling sensation to the skin or mucous membranes of the body, are well known and widely used. Examples of successful compounds include (1R,2S,5R)—N-ethyl-2-isopropyl-5-methylcyclohexane-carboxamide and 2-isopropyl-N,2,3-trimethylbutanamide, commercialised respectively as WS-3™ and WS-23™. A recent highly effective cooling compound is (1R,2S,5R)—N-(4-(cyanomethyl)phenyl)-2-isopropyl-5-methylcyclohexane-carboxamide, Evercool™ 180.

One problem with the cooling compounds has been the achievement of their maximum potential. Some cooling compounds do not dissolve well in solvents, which means that the quantity that can be added to some applications, and therefore the effect that can be perceived, is limited. Controlled release by means of encapsulation techniques has been suggested as a possible way of ameliorating this situation, but again there arises the problem of solubility with the solvents used in encapsulation. In addition, there can be problems with crystallisation of the compounds.

It has now been found that it is possible to considerably enhance the quantity that can be dissolved and therefore utilised in an application. There is therefore provided a liquid cooling composition, which is a mixture of at least one primary cooling compound, at least one different secondary cooling compound and at least one ingestible non-polar solvent for the primary cooling compound, the weight ratios of primary cooling compound:secondary cooling compound:solvent being 1:1.5-2.25:1.75-4.4.

There is additionally provided a method of incorporating at least one primary cooling compound in an application, comprising the blending of the primary cooling compound with at least one different secondary cooling compound and at least one ingestible non-polar solvent for the primary cooling compound, and the addition of the resulting mixture to the application.

By “liquid” is meant that the composition as formed is a liquid and remains thus, at least until incorporation into an end-product or into an entrapped form. It is a feature of the compositions that they can remain liquids for substantial periods of time at room temperature, varying from some hours to several weeks. In some cases, compositions can be held liquid at elevated temperature until processing. While crystallisation is not desirable in any circumstances, it is acceptable when it occurs when the composition has been incorporated into an end-product or into an entrapped form.

The composition has two different types of cooling compound, designated primary and secondary cooling compounds. By “primary” cooling compound is meant a cooling compound having a melting point of at least 75°, whereas a secondary cooling compound has a melting point of less than 90° C.

Some cooling compounds, for example, (1R,2S,5R)-2-isopropyl-5-methyl-N-(2-(pyridin-2-yl)ethyl)cyclohexanecarboxamide (Evercool™ 190), have a melting point that allows them to be either a primary or a secondary cooling compound. In such a case, the compound is selected to be either primary or secondary cooling compound and a different secondary or primary cooling compound is selected for blending with it.

Typical examples of primary cooling compounds include (1R,2S, 5R)—N-ethyl-2-isopropyl-5-methylcyclohexanecarboxamide, (1R,2S,5R)—N-(4-(cyanomethyl)phenyl)-2-isopropyl-5-methylcyclohexanecarboxamide (Evercool™ 180), (1R,2S,5R)-2-isopropyl-5-methyl-N-2-(pyridin-2-yl)ethyl)cyclohexanecarboxamide (Evercool™ 190).

Typical examples of secondary cooling compounds include menthyl lactate, 2-isopropyl-N,2,3-trimethylbutanamide (WS-23™), 1R,2S,5R)-2-isopropyl-5-methyl-N-(2-(pyridin-2-yl)ethyl)cyclohexanecarboxamide (Evercool™ 190).

The non-polar solvents for use herein are ingestible liquids, that is, they are solvents that may be used in foodstuffs, beverages and orally-receivable medicinal products. They are capable of dissolving the primary cooling compound to at least some extent, preferably to the extent of at least 0.0005 g/m L.

Typical examples of such solvents include Mygliol (MCT), peppermint oil, spearmint oil, triacetin, orange oil, lemon oil, lime oil, liquid flavors and menthol.

Particular compositions include:
(1R,2S,5R)—N-(4-(cyanomethyl)phenyl)-2-isopropyl-5-methylcyclohexanecarboxamide (Evercool™ 180)/menthyl lactate/Mygliol;
(1R,2S,5R)—N-(4-(cyanomethyl)phenyl)-2-isopropyl-5-methylcyclohexanecarboxamide (Evercool™ 180)/menthyl lactate/Triacetin:
(1R,2S,5R)—N-(4-(cyanomethyl)phenyl)-2-isopropyl-5-methylcyclohexanecarboxamide (Evercool™ 180)/2-isopropyl-N,2,3-trimethylbutanamide (WS-23™)/Mygliol;
(1R,2S,5R)-2-isopropyl-5-methyl-N-(2-(pyridin-2-yl)ethyl)cyclohexanecarboxamide (Evercool™ 190)/menthyl lactate/Mygliol:
(1R,2S,5R)-2-isopropyl-5-methyl-N-(2-(pyridin-2-yl)ethyl)cyclohexanecarboxamide (Evercool™ 190)/2-isopropyl-N,2,3-trimethylbutanamide (WS-23™)/Mygliol:
(1R,2S,5R)—N-(4-(cyanomethyl)phenyl)-2-isopropyl-5-methylcyclohexanecarboxamide (Evercool™ 180)/menthyl lactate/peppermint oil.
(1R,2S,5R)—N-(4-(cyanomethyl)phenyl)-2-isopropyl-5-methylcyclohexanecarboxamide (Evercool™ 180)/(1R,2S,5R)-2-isopropyl-5-methyl-N-(2-(pyridin-2-yl)ethyl)cyclohexanecarboxamide (Evercool™ 190)/menthyl lactate/Triacetin.

In a particular embodiment, the ratios of primary cooling compound:secondary cooling compound: solvent are 1:1.8-2.2:2.5-3.5.

The liquid compositions of this disclosure are exceptionally robust, some exceptionally so, having good shelf stability and exhibiting little or no precipitation, even under conditions of variable temperature and humidity. They may be easily incorporated into all manner of foodstuffs, beverages and orally-receivable medicinal products.

These compositions may be used in products that are applied to mucous membranes such as oral mucosa, or to the skin, to give a cooling sensation. By “applying” is meant any form of bringing into contact, for example, oral ingestion, topical application or, in the case of tobacco products, inhalation. In the case of application to the skin, it may be, for example, by including the compound in a cream or salve. There is therefore also provided a method of providing a cooling sensation to mucous membranes or skin by applying thereto a product comprising an effective amount of a cooling composition as hereinabove described.

Products that are applied to the oral mucosa include, but are not limited to foodstuffs and beverages taken into the mouth and swallowed, and products taken for reasons other than their nutritional value, e.g. tablets, troches, mouthwash, throat sprays, dentifrices and chewing gums, which may be applied to the oral mucosa for the purpose of cleaning, freshening, healing, and/or deodorising.

Products that are applied to the skin include, but are not limited to perfumes, toiletries, cosmetic products such as lotions, oils, ointments and bathing agents, applicable to the skin of the human body, whether for medical or other reasons.

Particular examples of foodstuffs and beverages include, but are not limited to frozen confectionery such as ice creams and sorbets; desserts such as jelly and pudding; confectionery such as cakes, cookies, chocolates, and chewing gum; jams; candies; breads: tea beverages such as green tea, black tea, chamomile tea, mulberry leaf tea. Roobos tea, peppermint tea: soups; seasonings; instant beverages; snack foods and the like.

Further examples of products for topical application may include, but are not limited to, skin-care cosmetics such as cleansing tissues, talcum powders, face creams, lotions, tonics and gels; hand creams, hand- and body lotions, anticellulite/slimming creams and -lotions, lotions, balms, gels, sprays and creams; sunburn cosmetics including sunscreen lotions, balms, gels, sprays and creams; after sun lotions, sprays and creams; soaps, toothpicks, lip sticks, agents for bathing, deodorants and antiperspirants, face washing creams, massage creams, and the like,

Further examples of products that are applied to the oral mucosa may include, but are not limited to, oral care products such as toothpastes, tooth gels, tooth powders, tooth whitening products, dental floss, anti-plaque and anti-gingivitis compositions, compositions for treatment of nasal symptoms, and the like.

Thus, there is further provided an end-product selected from the group consisting of products that are applied to the oral mucosa and products that are applied to the skin, such as products for topical application, oral care products, nasal care products, toilet articles, ingestible products and chewing gum, and the like, which comprises a product base and an effective amount of at least one cooling composition as hereinabove defined.

The cooling compositions hereinabove described may also be blended with known natural sensate compounds, for example, jambu, galangal, galangal acetate, sanshool, capscacian, pepper and ginger, or other flavour and fragrance ingredients generally known to the person skilled in the art. Suitable examples of flavour and fragrance ingredients include alcohols, aldehydes, ketones, esters, ethers, acetates, nitriles, terpene hydrocarbons, nitrogenous or sulphurous heterocyclic compounds. Flavor and fragrance ingredients may be of natural or synthetic origin. Many of these are listed in reference texts such as the book by S. Arctander, Perfume and Flavor Chemicals, 1969, Montclair, N.J. USA.

The cooling compositions may be employed in the products simply by directly mixing the composition with the product, or they may, in an earlier step, be entrapped in a suitable entrapment material. Suitable entrapment materials and means include, but are not limited to, polymer melts or hydrogels, capsules, microcapsules and nanocapsules, liposomes, film-formers and absorbents such as cyclic oligosaccharides (e.g. cyclodextrins). Alternatively, they may be added as a solution or emulsion in a suitable liquid, such liquids including alcohols or polyhydric alcohols (such as glycerine, propylene glycol) triacetin and Mygliol, using emulsifers or dispersion stabilizers such as natural gums (e.g., gum Arabic) or low molecular-weight surfactants, such as glycerine fatty acid esters and saccharide fatty acid esters.

The capsules may be provided by any convenient method, for example, core-shell complex coacervation capsules, matrix type hydrogel capsules, and coated capsules. Examples of commercially-available entrapment materials useful with these compositions include the ULTRASEAL™, GRANUSEAL™ AND QPEARL™ products of Givaudan Flavors Corp., but any other similar technology may also be used.

In a particular embodiment, the compositions are used in a spray-dried form. Spray-drying is a technology well known to the art. Typically it involves the emulsification of the composition into water in the presence of a suitable emulsifier, and then spraying the emulsion into a heated air stream. The result is a fine particulate material. The emulsifier may be any suitable emulsifier known to the art, typical examples including gum arabic, food-modified starch, maltodextrin, gum ghatti, gelatine, sodium caseinate, whey protein, milk protein, sodium alginate and citrus pectin.

There is therefore provided a composition as hereinabove defined, in spray dried form.

When entrapped compositions are used in products, they may be the sole source of cooling compounds, or they may be augmented with other cooling compounds known in the art, e.g. menthol, menthone, isopulegol, N-ethyl p-menthanecarboxamide (WS-3™), N,2,3-trimethyl-2-isopropylbutanamide, (WS-23™), ethyl 2-(2-isopropyl-5-methylcyclohexanecarboxamido)-acetate (WS-23™), menthyl lactate, menthone glycerine acetal (Frescolat® MGA), mono-menthyl succinate (Physcool®), mono-menthyl glutarate. O-menthyl glycerine (CoolAct® 10) and 2-sec-butylcyclohexanone (Freskomenthe®), menthane, camphor, pulegol, cineol, mint oil, peppermint oil, spearmint oil, eucalyptus oil, 3-1-menthoxypropane-1,2-diol, 3-1-menthoxy-2-methylpropane-1,2-diol, p-menthane-3,8-diol, 2-1-menthoxyethane-1-ol, 3-1-menthoxypropane-1-ol, 4-1-menthoxybutane-1-ol, and menthyl pyrrolidone carboxylic acid compounds sold under the commercial name Questice™. Further examples of cooling compounds can be found e.g. in WO 2005/049553 (e.g. 2-isopropyl-5-methyl-cyclohexanecarboxylic acid (4-cyanomethyl-phenyl)-amide and 2-isopropyl-5-methyl-cyclohexanecarboxylic acid (4-cyano-phenyl)-amide), WO2006/125334 (e.g. 4-[(2-isopropyl-5-methyl-cyclohexanecarbonyl)-amino]-benzamide, 3-[(2-isopropyl-5-methyl-cyclohexanecarbonyl)-amino]benzamide, and (2-isopropyl-5-methyl-N-(4-(4-methylpiperazine-1-carbonyl)phenyl)cyclohexane-carboxamide) and WO 2007/019719 (e.g. 2-isopropyl-5-methyl-cyclohexanecarboxylic acid pyridin-2-ylamide, and 2-isopropyl-5-methyl-cyclohexanecarboxylic acid (2-pyridin-2-yl-ethyl)-amide), which patent applications are incorporated herein by reference. These may be added conventionally in art-recognised quantities.

The compositions and methods are now further described with reference to the following non-limiting examples, which describe particular embodiments.

### EXAMPLE 1

Confirmation of suitable weight ratios of primary coolant (Evercool™ 180), secondary coolant (WS-23™) and solvent (Mygliol).

### General Procedure for Preparation of the Solutions:

Primary cooling agent was dissolved in molten secondary cooling agent and then diluted with solvent. The solution was allowed to cool to room temperature.

| Primary | | | | Solution | | | | |
|---|---|---|---|---|---|---|---|---|
| Coolant | Secondary | | | at RT | | | | |
| Evercool | Coolant | Solvent | Soluble | standing | Time at room temperature stirred | | | |
| 180 | WS-23 | Mygliol | hot | 30 min | 30 min | 60 min | 90 min | 180 min |
| 0.25 g | 0.25 g | 0.5 g | Yes | Yes | cryst. | cryst. | cryst. | cryst. |
| 0.25 g | 0.25 g | 0.6 g | Yes | Yes | cryst. | cryst. | cryst. | cryst. |
| 0.25 g | 0.25 g | 0.7 g | Yes | Yes | cryst. | cryst. | cryst. | cryst. |
| 0.25 g | 0.25 g | 0.8 g | Yes | Yes | cryst. | cryst. | cryst. | cryst. |
| 0.25 g | 0.25 g | 0.9 g | Yes | Yes | cryst. | cryst. | cryst. | cryst. |
| 0.25 g | 0.25 g | 1.0 g | Yes | Yes | cryst. | cryst. | cryst. | cryst. |
| 0.28 g | 0.42 g | 0.5 g | Yes | Yes | solution | solution | sl. | cryst. |
| | | | | | | | cloudy | |
| 0.28 g | 0.42 g | 0.6 g | Yes | Yes | solution | solution | sl. | cryst. |
| | | | | | | | cloudy | |
| 0.28 g | 0.42 g | 0.7 g | Yes | Yes | solution | solution | sl. | cryst. |
| | | | | | | | cloudy | |
| 0.28 g | 0.42 g | 0.8 g | Yes | Yes | solution | sl. | cryst. | cryst. |
| | | | | | | cloudy | | |
| 0.28 g | 0.42 g | 0.9 g | Yes | Yes | solution | sl. | cryst. | cryst. |
| | | | | | | cloudy | | |
| 0.28 g | 0.42 g | 1.0 g | Yes | Yes | cloudy | cryst. | cryst. | cryst. |
| 0.30 g | 0.60 g | 0.5 g | Yes | Yes | solution | sl. | cryst. | cryst. |
| | | | | | | cloudy | | |
| 0.30 g | 0.60 g | 0.6 g | Yes | Yes | solution | solution | solution | solution |
| 0.30 g | 0.60 g | 0.8 g | Yes | Yes | solution | solution | solution | solution |
| 0.30 g | 0.60 g | 0.9 g | Yes | Yes | solution | solution | solution | solution |
| 0.30 g | 0.60 g | 1.0 g | Yes | Yes | solution | solution | sl. | cloudy |
| | | | | | | | cloudy | |

The ratios primary coolant:secondary coolant:Mygliol of 1:1.5-2:2-3 are seen to be particularly effective.

### EXAMPLE 2

Primary coolant (WS-3™), secondary coolant (WS-23™) and solvent (Mygliol).

| Primary | Secondary | | | | | | | |
|---|---|---|---|---|---|---|---|---|
| Coolant | Coolant | Solvent | Soluble | Time at room temperature stirred | | | | |
| WS-3 | WS-23 | Mygliol | hot | 15 min | 30 min | 90 min | 180 min | 21 h |
| 0.33 g | 0.67 g | 1 g | Yes | solution | solution | solution | solution | solution |

### EXAMPLE 3

Primary coolant (Evercool™ 180), secondary coolant (menthyl lactate) and solvent (Mygliol).

| | Secondary | | | | |
|---|---|---|---|---|---|
| Primary | Coolant | | | Time at room temperature | |
| Coolant | Menthyl | Solvent | Soluble | standing | |
| Evercool 180 | lactate | Mygliol | hot | 15 min | 90 min |
| 0.03 g | 0.07 g | 1.0 g | Yes | crystallized | crystallized |
| 0.07 g | 0.13 g | 1.0 g | Yes | crystallized | crystallized |
| 0.10 g | 0.20 g | 1.0 g | Yes | crystallized | crystallized |
| 0.13 g | 0.27 g | 1.0 g | Yes | crystallized | crystallized |
| 0.17 g | 0.33 g | 1.0 g | Yes | crystallized | crystallized |
| 0.20 g | 0.40 g | 1.0 g | Yes | crystallized | crystallized |
| 0.23 g | 0.47 g | 1.0 g | Yes | solution | solution |
| 0.27 g | 0.53 g | 1.0 g | Yes | solution | solution |
| 0.30 g | 0.60 g | 1.0 g | Yes | solution | solution |
| 0.33 g | 0.67 g | 1.0 g | Yes | solution | sl. cloudy |
| 0.50 g | 1.00 g | 1.0 g | Yes | solution | sl. cloudy |
| 0.67 g | 1.33 g | 1.0 g | Yes | cloudy | crystallized |
| 1.00 g | 2.00 g | 1.0 g | Yes | solution | sl. cloudy |

Best Ratio for this Embodiment:

Primary coolant:secondary coolant:Mygliol 1:1.9-2.1:2-4.4

### EXAMPLE 4

Primary coolant (WS-3™), secondary coolant (menthyl lactate) and solvent (Mygliol).

| | Secondary | | | | | | | |
|---|---|---|---|---|---|---|---|---|
| Primary | Coolant | | | | | | | |
| Coolant | Menthyl | Solvent | Soluble | Time at room temperature stirred | | | | |
| WS-3 | lactate | Mygliol | hot | 15 min | 30 min | 90 min | 180 min | 21 h |
| 0.33 g | 0.67 g | 1 g | Yes | solution | solution | cloudy | cloudy | crystallized |

### EXAMPLE 5

Primary coolant (WS-3™), no secondary coolant and solvent (Mygliol).

| Primary | | | | | | | | |
|---|---|---|---|---|---|---|---|---|
| Coolant | Secondary | Solvent | Soluble | Time at room temperature stirred | | | | |
| WS-3 | Coolant | Mygliol | hot | 15 min | 30 min | 90 min | 180 min | 21 h |
| 0.33 g | — | 1.67 g | Yes | cryst. | cryst. | cryst. | cryst. | cryst. |

### EXAMPLE 6

Primary coolant (Evercool™ 180), secondary coolant (WS-3™) and solvent (Mygliol)-

Example of higher melting secondary coolant.

| Primary | Secondary | | | |
|---|---|---|---|---|
| Coolant | Coolant | Solvent | Soluble | |
| Evercool 180 | WS-3 | Mygliol | hot | |
| 1.0 g | 2.0 g | 3.0 g | Yes | Crystallized at ~50° C. |

### EXAMPLE 7

Determination of optimal concentrations in orange oil (1×, Brazilian) as solvent, using WS-23™ as secondary coolant and Evercool™ 180 as primary coolant.

| | Concentration |
|---|---|
| Primary coolant: Evercool 180 | x % |
| Secondary coolant: WS-23 | 2x % |
| Solvent: Orange oil 1x Brazil | 100 − 3x % |

| time | X % Primary Coolant (Secondary coolant = 2x %; solvent = 100 − 3x %) | | | | | | | | |
|---|---|---|---|---|---|---|---|---|---|
| [min] | 7% | 9% | 11% | 13% | 14% | 16% | 17% | 20% | 22% |
| 5 | sol. | sol. | sol. | sol. | | sol. | sol. | sol. | sol. |
| 10 | sol. | sol. | sol. | sol. | | sol. | sol. | sol. | sol. |
| 15 | sol. | sol. | sol. | sol. | | sol. | sol. | sol. | sol. |
| 20 | sol. | sol. | sol. | sol. | | sol. | sol. | sol. | sol. |
| 30 | cloudy | sol. | sol. | sol. | | sol. | sol. | sol. | sol. |
| 40 | cryst. | cloudy | sol. | sol. | | sol. | sol. | sol. | sol. |
| 50 | cryst. | cryst. | cloudy | sol. | | sol. | sol. | sol. | sol. |
| 60 | cryst. | cryst. | cryst. | cloudy | | sol. | sol. | sol. | sol. |
| 70 | cryst. | cryst. | cryst. | cryst. | cloudy | sol. | sol. | sol. | sol. |
| 80 | cryst. | cryst. | cryst. | cryst. | cryst. | cloudy | sol. | sol. | sol. |
| 90 | cryst. | cryst. | cryst. | cryst. | cryst. | cloudy | sol. | sol. | sol. |
| 105 | cryst. | cryst. | cryst. | cryst. | cryst. | cryst. | sol. | sol. | sol. |
| 120 | cryst. | cryst. | cryst. | cryst. | cryst. | cryst. | cloudy | cloudy | sol. |
| 180 | cryst. | cryst. | cryst. | cryst. | cryst. | cryst. | cryst. | cryst. | cloudy |
| 210 | cryst. | cryst. | cryst. | cryst. | cryst. | cryst. | cryst. | cryst. | cryst. |

Solubility significantly improved compared to >1% of Evercool™ 180 in Orange oil (1× Brazilian) under regular conditions.

### EXAMPLE 8

Determination of optimal concentrations in triactetin as solvent, using WS-23™ secondary coolant and Evercool™ 180 as primary coolant.

| | Concentration |
|---|---|
| Primary coolant: Evercool 180 | x % |
| Secondary coolant: WS-23 | 2x % |
| Solvent: Triacetin | 100 − 3x % |

| | x % Primary Coolant | | | | | |
|---|---|---|---|---|---|---|
| time | (Secondary coolant = 2x %; solvent = 100 − 3x %) | | | | | |
| [min] | 7% | 9% | 11% | 13% | 16% | 17% |
| 5 | sol. | sol. | sol. | sol. | sol. | sol. |
| 10 | sol. | sol. | sol. | sol. | sol. | sol. |
| 15 | sol. | sol. | sol. | sol. | sol. | sol. |
| 20 | sol. | sol. | sol. | sol. | sol. | sol. |
| 30 | sol. | sol. | sol. | sol. | sol. | sol. |
| 40 | sol. | sol. | sol. | sol. | sol. | sol. |
| 50 | sol. | sol. | sol. | sol. | sol. | sol. |
| 60 | sol. | sol. | sol. | sol. | sol. | sol. |
| 90 | sol. | sol. | sol. | sol. | sol. | sol. |
| 120 | sol. | sol. | sol. | sol. | sol. | sol. |
| 180 | sol. | sol. | sol. | sol. | sol. | sol. |
| 240 | sol. | sol. | sol. | sol. | sol. | sol. |
| 330 | sol. | sol. | cryst. | sol. | sol. | sol. |
| 420 | sol. | sol. | cryst. | sol. | sol. | sol. |
| 1440 | sol. | sol. | cryst. | sol. | sol. | cryst. |
| 1500 | cryst. | cryst. | cryst. | cryst. | cryst. | cryst. |

### EXAMPLE 9

Determination of optimal concentrations in menthol as solvent, using WS-23™ as secondary coolant and Evercool™ 180 as primary coolant.

| | Concentration |
|---|---|
| Primary coolant: Evercool 180 | x % |
| Secondary coolant: WS-23 | 2x % |
| Solvent: Menthol | 100 − 3x % |

| time | x % Primary Coolant (Secondary coolant = 2x %; solvent = 100 − 3x %) | | | | | | |
|---|---|---|---|---|---|---|---|
| [min] | 7% | 9% | 11% | 13% | 14% | 16% | 17% |
| 15 | sol. | sol. | sol. | sol. | sol. | sol. | sol. |
| 30 | sol. | sol. | sol. | sol. | sol. | sol. | sol. |
| 45 | sol. | sol. | sol. | sol. | sol. | sol. | sol. |
| 60 | sol. | sol. | sol. | sol. | sol. | sol. | sol. |
| 75 | cryst. | sol. | sol. | sol. | sol. | sol. | sol. |
| 90 | cryst. | sol. | sol. | sol. | sol. | sol. | sol. |
| 165 | cryst. | sol. | sol. | sol. | sol. | sol. | sol. |
| 180 | cryst. | sol. | sol. | sol. | sol. | sol. | sol. |
| 240 | cryst. | sol. | sol. | sol. | sol. | sol. | sol. |
| 300 | cryst. | cryst. | sol. | sol. | sol. | sol. | sol. |
| 1380 | cryst. | cryst. | sol. | sol. | sol. | cloudy | sol. |
| 1560 | cryst. | cryst. | sol. | sol. | cloudy | cryst. | sol. |
| 1740 | cryst. | cryst. | sol. | sol. | cryst. | cryst. | cloudy |
| 2880 | cryst. | cryst. | sol. | cryst. | cryst. | cryst. | cryst. |
| 3180 | cryst. | cryst. | cloudy | cryst. | cryst. | cryst. | cryst. |

Solubility significantly improved, compared to 17% Evercool™ 180 soluble in menthol at 65° C. under regular conditions

### EXAMPLE 10

Determination of optimal concentrations in Orange Oil 10× California as solvent, using WS-23™ as secondary coolant and Evercool 180 as primary coolant

| | Concentration |
|---|---|
| Primary coolant: Evercool 180 | x % |
| Secondary coolant: WS-23 | 2x % |
| Solvent: Orange Oil 10x California | 100-3x % |

| | |
|---|---|
| | x % Primary Coolant (Secondary |
| time | coolant = 2x %: solvent = 100-3x %) |

| [min] | 7% | 11% | 14% | 17% | 20% |
|---|---|---|---|---|---|
| 10 | sol. | sol. | sol. | sol. | sol. |
| 20 | sol. | sol. | sol. | sol. | sol. |
| 30 | sol. | sol. | sol. | sol. | sol. |
| 40 | sol. | sol. | sol. | sol. | sol. |
| 50 | sol. | sol. | sol. | sol. | sol. |
| 60 | sol. | sol. | sol. | sol. | sol. |
| 105 | sol. | sol. | sol. | sol. | sol. |
| 180 | sol. | sol. | sol. | sol. | sol. |
| 285 | sol. | cryst. | cryst. | cloudy | sol. |
| 345 | sol. | cryst. | cryst. | cryst. | cloudy |
| 360 | sol. | cryst. | cryst. | cryst. | cloudy |
| 1200 | cloudy | cryst. | cryst. | cryst. | cryst. |

### EXAMPLE 11

Determination of optimal concentrations in Peppermint oil as solvent, using WS-23™ as secondary coolant and Evercool™ 180 as primary coolant.

| | Concentration |
|---|---|
| Primary coolant: Evercool 180 | x % |
| Secondary coolant: WS-23 | 2x % |
| Solvent: Peppermint oil | 100 − 3x % |

| time | x % Primary Coolant (Secondary coolant = 2x %; solvent = 100 − 3x %) | | | | | | | | | | |
|---|---|---|---|---|---|---|---|---|---|---|---|
| [min] | 7% | 9% | 11% | 13% | 14% | 16% | 17% | 18% | 19% | 19% | 20% |
| 30 | sol. | sol. | sol. | sol. | sol. | sol. | sol. | sol. | sol. | sol. | sol. |
| 60 | sol. | sol. | sol. | sol. | sol. | sol. | sol. | sol. | sol. | sol. | sol. |
| 120 | sol. | sol. | sol. | sol. | sol. | sol. | sol. | sol. | sol. | sol. | sol. |
| 180 | sol. | sol. | sol. | sol. | sol. | sol. | sol. | sol. | sol. | sol. | sol. |
| 240 | sol. | sol. | sol. | sol. | sol. | sol. | sol. | sol. | sol. | sol. | sol. |
| 300 | sol. | sol. | sol. | sol. | sol. | sol. | sol. | sol. | sol. | sol. | sol. |
| 360 | sol. | sol. | sol. | sol. | sol. | sol | sol. | sol. | sol. | sol. | sol. |
| 1440 | sol. | sol. | sol. | sol. | sol. | sol. | sol. | sol. | sol. | sol. | sol. |
| 2640 | sol. | sol. | sol. | sol. | sol. | sol. | sol. | sol. | sol. | sol. | cloudy |
| 2 weeks | sol. | sol. | sol. | sol. | sol. | sol. | sol. | sol. | cryst. | cryst. | cryst. |
| 6 weeks | sol. | sol. | sol. | sol. | sol. | sol. | sol. | sol. | cryst. | cryst. | cryst. |

Solubility significantly improved, compared to 12% Evercool™ 180 soluble in peppermint oil at room temperature under regular conditions.

### EXAMPLE 12

Determination of optimal concentrations in polar solvent, Propylene glycol, using WS-23™ as secondary coolant and Evercool™ 180 as primary coolant.

| | Concentration |
|---|---|
| Primary coolant: Evercool 180 | x % |
| Secondary coolant: WS-23 | 2x % |
| Solvent: Propylene glycol | 100 − 3x % |

| time | x % Primary Coolant (Secondary coolant = 2x %; solvent = 100 − 3x %) | | | | | | | | |
|---|---|---|---|---|---|---|---|---|---|
| [min] | 7% | 9% | 11% | 13% | 14% | 16% | 17% | 20% | 22% |
| 5 | cryst. | cryst. | cryst. | cryst. | cryst. | cryst. | cryst. | cryst. | cryst. |

### EXAMPLE 13

Primary coolant (Evercool™ 180) secondary coolant (WS-5™), solvent (Mygliol), 1 g of Evercool™ 180 was dissolved hot in 2 g of WS-5™ and then 3 g of Mygliol were added. The solution was allowed to cool to room temperature under stirring and became cloudy only after 3 h, crystallized alter 4 h.

### EXAMPLE 14

Primary coolant (Evercool™ 180) secondary coolant ((1R,2S,5R)-2-isopropyl-5-methyl-N-phenethylcyclohexanecarboxamide), solvent (Mygliol) 1 g of Evercool180 was dissolved hot in 2 g of (1R,2S,5R)-2-isopropyl-5-methyl-N-phenethylcyclohexanecarboxamide and then 3 g of Mygliol were added. The solution was allowed to cool to room temperature under stirring and became cloudy after 1 h, crystallized after 6 h.

### EXAMPLE 15

Encapsulation of a mixture of Menthyl Lactate (secondary coolant) and Evercool 180 (primary coolant) and Mygliol (solvent) in a non-cariogenic spray dry matrix:

The coolants may be solubilized in an oil phase before they are added to the spray dry matrix to form an emulsion. For this system, the 32 g of menthyl lactate were melted above 60° C. 16 g of Evercool 180 was added to this (2:1 weight ratio of menthyl lactate to Evercool 180) and heated to above 100° C. until solubilized, 48 g of hot Mygliol (above 100° C.) was then added to this homogeneous solution at a 1:1 ratio. The solution then was held above 80° C. to maintain solubility. This oil phase was then added to a gum arabic solution (36% solution in water, 400 g gum arabic, 700 g water) which was held at 60° C. This emulsion was then spray dried under normal conditions.

### EXAMPLE 16

Application of spray-dried mixture of Menthyl Lactate (secondary coolant) and Evercool™ 180 and comparison to references:

| | | |
|---|---|---|
| Gum Base Solsona-T | 30 | g |
| Sorbitol powdered | 50.6 | g |
| Maltitol Syrup 85% | 9 | g |
| Mannitol powdered | 5 | g |
| Glycerin | 5 | g |
| Acesulfame potassium (Ace-K ™) | 0.09 | g |
| Aspartame | 0.21 | g |

Coolant mix spray dried (see amounts in table below)

| | | |
|---|---|---|
| 0.32 g | Evercool 180 (spray dried with menthyl | Delivering 100 ppm of |
| | lactate; 3.2% load | Evercool 180 |
| 0.10 g | Evercool 180 (spray dried without | Delivering 100 ppm of |
| | menthyl lactate; 10% load) [Reference] | Evercool 180 |
| 0.20 g | Menthyl lactate (spray dried; 10% load) | Delivering 200 ppm of |
| | [Reference] | Menthyl lactate |

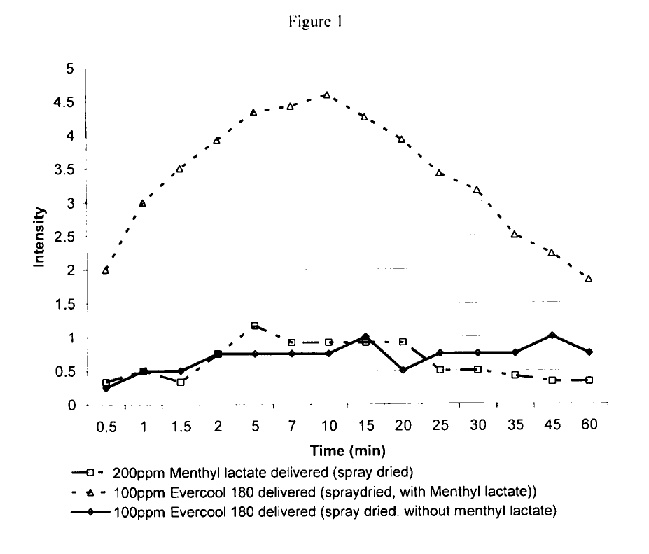

The gum base, and half of the sorbitol were mixed, maltitol syrup was added and then mixed with the gum mass. The rest of the powdered ingredients (rest of the sorbitol, mannitol, ace-K, aspartame) were added and mixed for about 1 minute, at which point glycerine was added and the gum mass was mixed for about 5 minutes, to form the blank chewing gum mass. The coolant mix was worked into the mass and a piece of the resulting gum (2 g) was chewed by a panelist for 20 min and spat out. The cooling sensation was evaluated and its time intensity profile were recorded (see FIG. 1).

### EXAMPLE 17

Encapsulation of a mixture of Menthyl Lactate (secondary coolant) and Evercool 180 (primary coolant) and Mygliol (solvent) in an encapsulation matrix:

The coolants must be solubilized in an oil phase before they are added to the spray dry matrix to form an emulsion. For this system, the 50 g of menthyl lactate were melted above 60° C. 25 g of Evercool 180 was added to this (2:1 weight ratio of menthyl lactate to Evercool 180) and heated to above 100° C. until solubilized and added to 95 g of hot Mygliol (above 75° C.). This solution then was added to a hot (75° C.) solution of 132 g of Gelatin 100, 42 g of Gelatin 0.13 g of Potato starch and 43 g of Xylitol in 340 g of Water and homogenized. The solution was kept stirring at 75° C. during coating onto 100 g of Gelatin 100 using a Wurster insert (Tₛₒₗᵤₜᵢₒₙ:75° C., P_{nozzle}:2.5 bar. Tᵢₙₗₑₜ:90° C.).

### EXAMPLE 18

Application of encapsulated mixture of Menthyl Lactate (secondary coolant) and Evercool™ 180 and comparison to references:

| | | |
|---|---|---|
| Gum Base Solsona-T | 30 | g |
| Sorbitol powdered | 50.6 | g |
| Maltitol Syrup 85% | 9 | g |
| Mannitol powdered | 5 | g |
| Glycerin | 5 | g |
| Acesulfame potassium (Ace-K ™) | 0.09 | g |
| Aspartame | 0.21 | g |

Coolant mix encapsulated or spray dried (see amounts in table below)

| | | |
|---|---|---|
| 0.20 g | Evercool 180 (encapsulated with | Delivering 100 ppm of |
| | menthyl lactate; 5% load | Evercool 180 |
| 0.20 g | Menthyl lactate (spray dried; 10% load) | Delivering 200 ppm of |
| | [Reference] | Menthyl lactate |

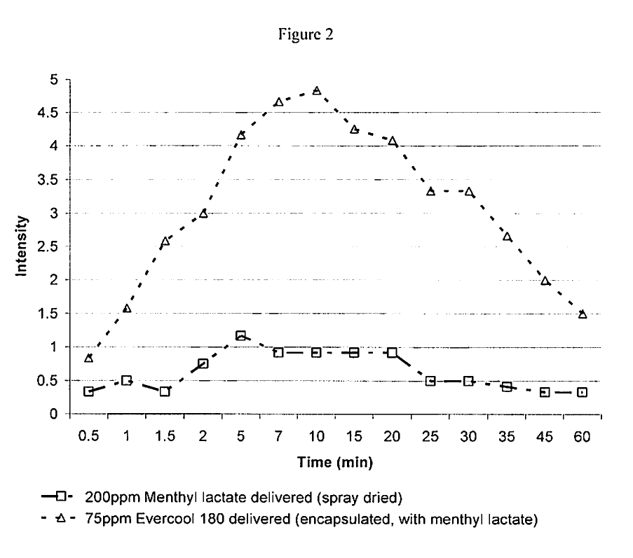

The gum base, and half of the sorbitol were mixed, maltitol syrup was added and then mixed with the gum mass. The rest of the powdered ingredients (rest of the sorbitol, mannitol, ace-K, aspartame) were added and mixed for about 1 minute, at which point glycerine was added and the gum mass was mixed for about 5 minutes, to form the blank chewing gum mass. The coolant mix was worked into the mass and a piece of the resulting gum (2 g) was chewed by a panelist for 20 min and spat out. The cooling sensation was evaluated and its time intensity profile were recorded (see FIG. 2).

It will be understood that the embodiments described herein are merely exemplary and that variations and modifications can be made by one skilled in the art without departing from the scope of the invention. It should be understood that the embodiments described above are not only in the alternative, but can be combined.

1. A liquid cooling composition, which is a mixture of at least one primary cooling compound, at least one different secondary cooling compound and at least one ingestible non-polar solvent for the primary cooling compound, the weight ratios of primary cooling compound:secondary cooling compound:solvent being 1:1.5-2.25:1.75-4.4.

2. A composition according to claim 1, in which the weight ratios of primary cooling compound:secondary cooling compound:solvent are 1:1.8-2.2:2.5-3.5.

3. A composition according to claim 1, in which the primary cooling compound is selected from the group consisting of (1R,2S,5R)—N-ethyl-2-isopropyl-5-methylcyclohexanecarboxamide, (1R,2S,5R)—N-(4-(cyanomethyl)phenyl)-2-isopropyl-5-methylcyclohexanecarboxamide, and (1R,2S,5R)-2-isopropyl-5-methyl-N-(2-(pyridin-2-yl)ethyl)cyclohexanecarboxamide, and mixtures thereof.

4. A composition according to claim 1, in which the secondary cooling compound is selected from the group consisting of menthyl lactate, 2-isopropyl-N,2,3-trimethylbutanamide, and (1R,2S,5R)-2-isopropyl-5-methyl-N-(2-(pyridin-2-yl)ethyl)cyclohexanecarboxamide, and mixtures thereof.

5. A composition according to claim 1, in which the non-polar solvent is capable of dissolving the primary cooling compound to the extent of at least 0.0005 g/mL.

6. A composition according to claim 1, in which the primary cooling compound/secondary cooling compound/solvent combination is selected from the group consisting of:
(1R,2S,5R)—N-(4-(cyanomethyl)phenyl)-2-isopropyl-5-methylcyclohexane-carboxamide/menthyl lactate/Mygliol;
(1R,2S,5R)—N-(4-(cyanomethyl)phenyl)-2-isopropyl-5-methylcyclohexane-carboxamide/menthyl lactate/Triacetin;
(1R,2S,5R)—N-(4-(cyanomethyl)phenyl)-2-isopropyl-5-methylcyclohexane-carboxamide/2-isopropyl-N,2,3-trimethylbutanamide/Mygliol;
(1R,2S,5R)-2-isopropyl-5-methyl-N-(2-(pyridin-2-yl)ethyl)cyclohexane-carboxamide/menthyl lactate/Mygliol:
(1R,2S,5R)-2-isopropyl-5-methyl-N-(2-(pyridin-2-yl)ethyl)cyclohexane-carboxamide/2-isopropyl-N,2,3-trimethylbutanamide/Mygliol;
(1R,2S,5R)—N-(4-(cyanomethyl)phenyl)-2-isopropyl-5-methylcyclohexane-carboxamide/menthyl lactate/peppermint oil; and
(1R,2S,5R)—N-(4-(cyanomethyl)phenyl)-2-isopropyl-5-methylcyclohexanecarboxamide (Evercool™ 180)/(1R,2S,5R)-2-isopropyl-5-methyl-N-(2-(pyridin-2-yl)ethyl)cyclohexanecarboxamide (Evercool™ 190)/menthyl lactate/Triacetin.

7. A product adapted to be applied to at least one or the oral mucosa and the skin, which comprises a product base and an effective amount of at least one cooling composition according to claim 1.

8. A product according to claim 7, in which the cooling composition is present in an entrapped form.

9. A product according to claim 8, in which the entrapped form is selected from the group consisting of polymer melts or hydrogels, capsules, microcapsules and nanocapsules, liposomes, film-formers and absorbents.

10. A product according to claim 8, in which the entrapped form is a spray-dried granulate.

11. A method of incorporating at least one primary cooling compound in a product, comprising the blending of the primary cooling compound with at least one different secondary cooling compound and at least one ingestible non-polar solvent for the primary cooling compound, and the addition of the resulting mixture to the product.
